(19) United States
(12) Patent Application Publication
(10) Pub. No.: US 2008/0120135 A1
(43) Pub. Date: May 22, 2008

(54) ABSTRACTION OF COMMUNICATION PROTOCOLS FOR PACS COLLABORATION SOFTWARE

(75) Inventors: Sukhdeep Gill, London (CA); Mark M. Morita, Arlington Heights, IL (US); Prakash Mahesh, Hoffman Estates, IL (US); Shuo Li, London (CA)

Correspondence Address: MCANDREWS HELD & MALLOY, LTD, 500 WEST MADISON STREET, SUITE 3400, CHICAGO, IL, 60661, US

(73) Assignee: GENERAL ELECTRIC COMPANY, Schenectady, NY (US)

(21) Appl. No.: 11/845,392

(22) Filed: Aug. 27, 2007

Related U.S. Application Data
(60) Provisional application No. 60/866,996, filed on Nov. 22, 2006.

(51) Int. Cl. G06Q 50/00; A61B 5/00

(52) U.S. Cl. 705/2; 600/300

(57) ABSTRACT

Certain embodiments of the present invention provide methods and systems for collaboration between systems in a healthcare environment. Certain embodiments provide a collaboration framework enabling electronic communication in a healthcare environment. The framework includes a plurality of communication abstraction layers, each of the plurality of communication abstraction layers defining interface functionality for a communication method. In certain embodiments, each of the plurality of communication abstraction layers includes a general set of features and actions. In certain embodiments, the general set of features and actions is representative of features and actions for a specific protocol or technology. The method includes transmitting data from a first system to a second system, abstracting data format and/or protocol to a general format and/or protocol and receiving the abstracted data at the second system.

### RELATED APPLICATIONS

This application relates to and claims the benefit of priority from U.S. Provisional Application No. 60/866,996, filed on Nov. 22, 2006, which is hereby incorporated in its entirety herein.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to healthcare system collaboration and workflow. In particular, the present invention relates to use of communication abstractions to facilitate collaboration between healthcare systems.

Various communication methods and protocols exist for electronic communication, ranging from email to messaging to Voice over IP (VoIP) protocols. Today, healthcare enterprises have designed and implemented their communication infrastructures with a vast number of technologies. Today's healthcare communication infrastructures result from work with various specialist vendors that implement systems customized for a particular enterprise. For example, Healthcare Enterprise #1 implements VoIP System ‘XYZ’, Instant Messaging (IM) System ‘ABC’, and Mail System ‘123’. Healthcare Enterprise #2 implements VOIP System ‘ZYX’, IM System ‘CBA’, and Mail System ‘321’.

Management of multiple and disparate devices and systems in a diverse and crowded healthcare environment is difficult for medical or healthcare personnel. Additionally, a lack of interoperability between devices and systems increases delay and inconvenience associated with their use in a healthcare workflow. A system and method for improving ease of use and interoperability between multiple systems in a healthcare environment would be highly desirable.

For example, healthcare environments, such as hospitals or clinics, include a plurality of clinical information systems, such as hospital information systems (HIS), radiology information systems (RIS), clinical information systems (CIS), and cardiovascular information systems (CVIS), and storage systems, such as picture archiving and communication systems (PACS), library information systems (LIS), and electronic medical records (EMR). Information stored may include patient medical histories, imaging data, test results, diagnosis information, management information, and/or scheduling information, for example. The information may be centrally stored or divided among a plurality of locations. Healthcare practitioners may desire to access patient information or other information at various points in a healthcare workflow. For example, during surgery, medical personnel may access patient information, such as images of a patient's anatomy, that are stored in a medical information system. Alternatively, medical personnel may enter new information, such as history, diagnostic, or treatment information, into a medical information system during an ongoing medical procedure. Systems may communicate and/or be accessed using a variety of protocols and technologies.

Imaging systems are complicated to configure and to operate. Often, healthcare personnel may be trying to obtain an image of a patient, reference or update patient records or diagnosis, and/or ordering additional tests or consultation, for example. Thus, there is a need for a system and method that facilitate operation and interoperability of an imaging system and related devices by an operator.

Additionally, in a healthcare workflow, healthcare providers often consult or otherwise interact with each other. Such interaction typically involves paging or telephoning another practitioner. Thus, interaction between healthcare practitioners may be time- and energy-consuming. Therefore, there is a need for a system and method to simplify and improve communication and interaction between healthcare practitioners.

Furthermore, healthcare practitioners may want or need to review diagnoses and/or reports from another healthcare practitioner. For example, a referring physician may want to review a radiologist's diagnosis and report with the radiologist and/or a technician. As another example, an emergency room physician may need to review results of an emergency room study with the radiologist and/or a family physician. Thus, there is a need for a system and method for notifying or informing appropriate parties of results in order to collaborate for diagnosis and/or treatment review for safe and effective treatment.

Healthcare experts are located around the world and are often separated by large distances. Collaboration between experts and other healthcare practitioners is often difficult to coordinate. Additionally, current collaboration systems and efforts often operate using different technologies and/or protocols and are difficult to coordinate under a single framework. Thus, there is a need for a system and method for improved collaboration and control in a healthcare environment.

As a collaboration system attempts to incorporate various features of the existing communications infrastructure, the collaboration system should support the healthcare enterprise's choice of technology/solution. For integration, the collaboration system should implicitly implement an interface(s) to communicate with each technology/solution. Such interfacing results in developing a unique interface for each healthcare enterprise.

Thus, there is a need for systems and methods for healthcare collaboration supporting a variety of underlying communication technologies and protocols. There is a need for systems and methods for communication protocol abstraction in a healthcare environment.

### BRIEF SUMMARY OF THE INVENTION

Certain embodiments of the present invention provide methods and systems for collaboration between systems in a healthcare environment. Certain embodiments provide a collaboration framework enabling electronic communication in a healthcare environment. The framework includes a plurality of communication abstraction layers, each of the plurality of communication abstraction layers defining interface functionality for a communication method. In certain embodiments, each of the plurality of communication abstraction layers includes a general set of features and actions. In certain embodiments, the general set of features and actions is representative of features and actions for a specific protocol or technology. In certain embodiments, the plurality of communication abstraction layers includes at least one of a voice over internet protocol abstraction, an instant messaging abstraction and a presence management abstraction. In certain embodiments, each of the voice over internet protocol abstraction, instant messaging abstraction and presence management abstraction accommodates a plurality of specific features and actions for a plurality of technology-specific solutions. Certain embodiments provide a method for abstracted communication in a healthcare environment. The method includes transmitting data from a first system to a second system, abstracting data format and/or protocol to a general format and/or protocol and receiving the abstracted data at the second system.

Certain embodiments provide a computer readable medium having a set of instructions for execution by a computer. The set of instructions includes a communication framework interface routine proving a general set of features and actions to facilitate access to a plurality of healthcare enterprise communications applications. The set of instructions also includes a plurality of communication abstraction layers accessible via the communication framework interface, each of the plurality of communication abstraction layers defining interface functionality for a communication application.

### BRIEF DESCRIPTION OF SEVERAL VIEWS OF THE DRAWINGS

FIG. 1 illustrates a communication framework system in accordance with an embodiment of the present invention.

FIG. 2 illustrates a flow diagram for a method for abstracted communication in a healthcare environment in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

Certain embodiments provide a healthcare collaboration framework for interfacing and connecting a plurality of underlying healthcare enterprise communications infrastructure. For purposes of illustration only, the following description is given in terms of an exemplary Picture Archiving and Communication System (PACS) collaboration framework. However, it is understood that the healthcare collaboration framework may include one or more healthcare systems, including a PACS, Radiology Information System (RIS), Cardiovascular Information System (CVIS), Library Information System (LIS), Hospital Information System (HIS), etc.

In a PACS collaboration framework, abstraction of one or more interface(s) to various system implementations can simplify a communication system design and allow for greater protocol support. In certain embodiments, a PACS collaboration framework system defines a general set of features and actions. Implementation of the features and actions depends on the respective protocol/technology being used. In certain embodiments, an Abstract layer is created to define a communication method and its functions. Implementation of a method's/functions may then be protocol/vendor and/or technology specific, for example.

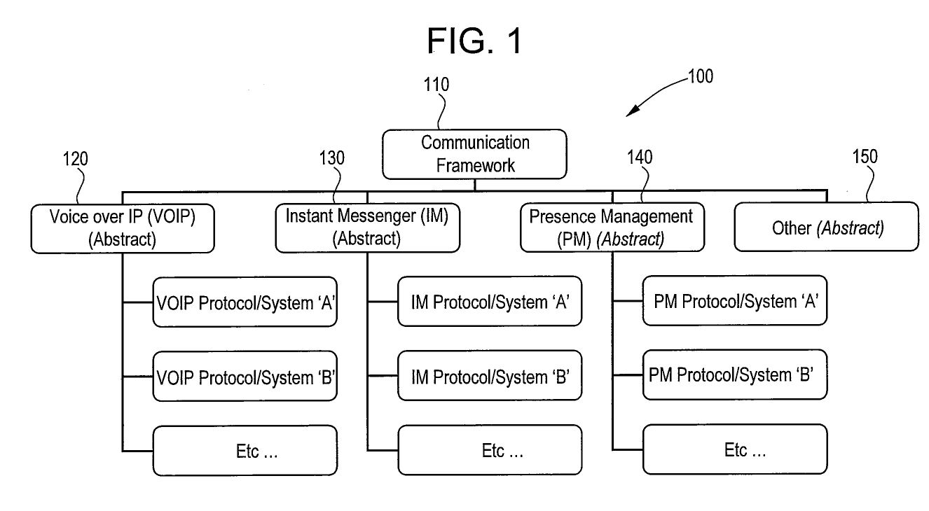

FIG. 1 illustrates a communication framework system 100 in accordance with an embodiment of the present invention. The communication framework 110 a superset of various types of electronic communication. For example, the communication framework 110 may provide an interface to one or more of a Voice over IP (VOIP) application, instant messaging (IM) application, presence management (PM) application, etc., via a VOIP abstraction 120, an IM abstraction 130, a PM abstraction 140 and/or other abstraction 150.

For each abstraction 120, 130, 140 and/or 150, a particular technology solution implements a set of general functionality to communicate with and/or execute an application, such as VOIP communication; instant messaging of text, images and/or other data; determination of a presence or logon of a user in the system; etc., rather than providing a set of functionality for each vendor-specific technology solution. Thus, certain embodiments provide a robust, adaptable solution that can be used with many different site/enterprise systems.

The communication framework 110 provides an abstraction on top of a plurality of communication protocols/technologies (e.g., VOIP, instant message, etc.). The abstraction standardizes inputs and output for an application or service such that a general set of functions (e.g., a common application program interface or API) may be used to facilitate communication and execution, for example. The framework 110 provides an abstract layer that can communicate with a plurality of lower-level objects. For example, suppose that one part of a hospital communicates via a Cisco network, and another part of the hospital uses a Nortel network for data communication. Depending upon which part of the hospital they are in, users are currently tying into a particular engine for data communication on a particular network. The communication framework 110 provides an abstraction for particular communication engine(s) and automatically ties in to an appropriate network without knowledge of and/or special interaction by the user. Thus, certain embodiments provide an abstraction layer such that a VOIP application, screen sharing application, instant message application, and/or any disparate application from a wide variety of vendors may be accommodated transparently to a user.

In certain embodiments, a plurality of communication systems are connected in a healthcare enterprise via wired, wireless and/or infrared communication, for example. The communication systems may be separate systems and/or may be integrated in various forms, for example. The systems may be implemented in software, hardware and/or firmware, for example. The systems transmit and/or receive information via a communication medium/device, such as a wired or wireless modem, cellular transmission, infrared transmission, Ethernet, fire wire, Internet, virtual private network, public switched telephone network, dial-up, local area network, and/or wide area network, for example.

The one or more abstractions 120, 130, 140, 150 provide general interfaces for one or more underlying protocols/systems to communicate via the common communication framework 110 without regard to particular protocol and/or technology. For example, as shown in FIG. 1, the VOIP abstraction 120 may provide an abstraction layer to allow VOIP protocol/system A, VOIP protocol/system B, etc. to communicate with each other and/or with other components of the system 100 via the abstraction 120 and framework 110. Thus, a user on system A may talk with a user on system B via the abstraction layer 120, for example.

As another example, the IM abstraction 130 may provide an abstraction layer to allow IM protocol/system A, IM protocol/system B, etc. to communicate with each other and/or with other components of the system 100. Thus, a user on system A may send a message to a user on system B via the abstraction layer 130, for example.

As another example, the PM abstraction 140 may provide an abstraction layer to allow PM protocol/system A, PM protocol/system B, etc. to communicate with each other and/or with other components of the system 100. Thus, system A and system B may communicate to look for a “presence” of a user across systems A and B via the abstraction layer 130, for example. That is, systems A and B may communicate to determine whether a user is registered or “logged in” in the healthcare environment. For example, system A and/or B may determine whether the referring physician has his or her cellular phone switched on. In an embodiment, a user may set presence parameters, such as available, away, busy, do not disturb except for certain occurrence(s), etc. For example, a surgeon may set his or her status to do not disturb except for emergencies when in surgery. In certain embodiments, systems A and B may communicate via abstraction 140 to set meetings, conferences, data exchange, etc.

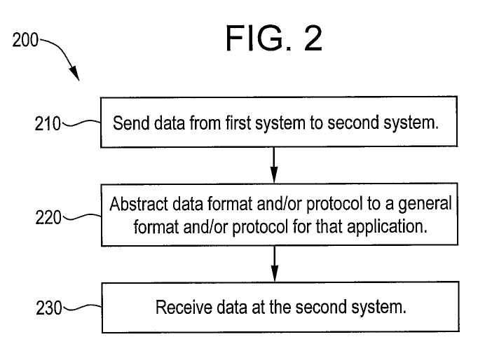

FIG. 2 illustrates a flow diagram for a method 200 for abstracted communication in a healthcare environment in accordance with an embodiment of the present invention. At step 210, data is sent from a first system to a second system in a healthcare environment. For example, a text message is sent from a user of IM system A to a user of IM system B. Data may include healthcare data, one or more application commands, and/or an application for execution at and/or with the second system, for example.

At step 220, data format and/or transmission/reception protocol is abstracted to a general format and/or protocol via an abstraction layer. For example, an appropriate abstraction is selected, and the text message from system A to system B is formatted and transmitted according to a general IM abstraction definition. Such formatting/translation may occur automatically without user intervention, for example.

At step 230, data is received at the second system. For example, the text message is received in the general format at system B and converted for display to the user at system B. The abstracted data may be translated to a specific format and/or protocol for the second system, for example. Such formatting/translation may occur automatically without user intervention, for example.

One or more of the steps of the method 200 may be implemented alone or in combination in hardware, firmware, and/or as a set of instructions in software, for example. Certain embodiments may be provided as a set of instructions residing on a computer-readable medium, such as a memory, hard disk, DVD, or CD, for execution on a general purpose computer or other processing device.

Certain embodiments of the present invention may omit one or more of these steps and/or perform the steps in a different order than the order listed. For example, some steps may not be performed in certain embodiments of the present invention. As a further example, certain steps may be performed in a different temporal order, including simultaneously, than listed above.

Certain embodiments may be implemented in a computer readable medium having a set of instructions for execution by a computer, for example. The computer-readable medium and its instructions may be used to execute the communication abstraction framework as described above. In certain embodiments, the set of instructions may include a communication framework interface routine proving a general set of features and actions to facilitate access to a plurality of healthcare enterprise communications applications, for example. The set of instructions may also include a plurality of communication abstraction layers accessible via the communication framework interface, each of the plurality of communication abstraction layers defining interface functionality for a communication application, for example.

Thus, certain embodiments provide integration of communication capabilities in a healthcare environment. Certain embodiments provide communications capabilities and functions as an adjunct to more efficient workflow and enhanced ability to deliver healthcare services. Certain embodiments provide a technical effect of allowing abstracted communication between various components and subsystems in a healthcare environment.

The components, elements, and/or functionality of the interface(s) and system(s) described above may be implemented alone or in combination in various forms in hardware, firmware, and/or as a set of instructions in software, for example. Certain embodiments may be provided as a set of instructions residing on a computer-readable medium, such as a memory or hard disk, for execution on a general purpose computer or other processing device, such as, for example, a PACS workstation or one or more dedicated processors.

Several embodiments are described above with reference to drawings. These drawings illustrate certain details of specific embodiments that implement the systems and methods and programs of the present invention. However, describing the invention with drawings should not be construed as imposing on the invention any limitations associated with features shown in the drawings. The present invention contemplates methods, systems and program products on any machine-readable media for accomplishing its operations. As noted above, the embodiments of the present invention may be implemented using an existing computer processor, or by a special purpose computer processor incorporated for this or another purpose or by a hardwired system.

As noted above, certain embodiments within the scope of the present invention include program products comprising machine-readable media for carrying or having machine-executable instructions or data structures stored thereon. Such machine-readable media can be any available media that can be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

Certain embodiments of the invention are described in the general context of method steps which may be implemented in one embodiment by a program product including machine-executable instructions, such as program code, for example in the form of program modules executed by machines in networked environments. Generally, program modules include routines, programs, objects, components, data structures, etc., that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

Certain embodiments of the present invention may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer.

The foregoing description of embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principals of the invention and its practical application to enable one skilled in the art to utilize the invention in various embodiments and with various modifications as are suited to the particular use contemplated.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A collaboration framework enabling electronic communication in a healthcare environment, said framework comprising:
a communication framework interface providing a general set of features and actions to facilitate access to a plurality of healthcare enterprise communications applications; and
a plurality of communication abstraction layers accessible via said communication framework interface, each of said plurality of communication abstraction layers defining interface functionality for a communication application.

2. The framework of claim 1, wherein each of said plurality of communication abstraction layers comprises a general set of features and actions.

3. The framework of claim 2, wherein said general set of features and actions is representative of features and actions for a specific protocol or technology.

4. The framework of claim 1, wherein said plurality of communication abstraction layers include at least one of a voice over internet protocol abstraction, an instant messaging abstraction and a presence management abstraction for at least one of a voice over internet protocol application, an instant messaging application and a presence management application, respectively.

5. The framework of claim 4, wherein each of said voice over internet protocol abstraction, instant messaging abstraction and presence management abstraction layers accommodates a plurality of specific features and actions for a plurality of technology-specific solutions.

6. The framework of claim 5, wherein each of said voice over internet protocol abstraction, instant messaging abstraction and presence management abstraction layers translate between a general set of features and actions for an application and a specific set of features and actions for a specific solution.

7. The framework of claim 6, wherein said translation occurs automatically between networks without additional input from a user.

8. A method for abstracted communication in a healthcare environment, said method comprising:
transmitting data from a first system to a second system, the data formatted according to at least one of a first format and a first protocol;
selecting an abstraction for the data from among a plurality of communication abstraction layers according to a function of the first system and the second system;
abstracting at least one of the first format and first protocol for the data according to the selected abstraction to form abstracted data in at least one of a general format and general protocol; and
receiving the abstracted data at the second system.

9. The method of claim 8, wherein the data comprises at least one of healthcare data, one or more application commands, and an application.

10. The method of claim 8, further comprising translating the abstracted data according to at least one of a second format and a second protocol for the second system.

11. The method of claim 8, wherein each of said plurality of communication abstraction layers comprises a general set of features and actions.

12. The method of claim 11, wherein said general set of features and actions is representative of features and actions for a specific protocol or technology.

13. The method of claim 8, wherein said plurality of communication abstraction layers include at least one of a voice over internet protocol abstraction, an instant messaging abstraction and a presence management abstraction for at least one of a voice over internet protocol application, an instant messaging application and a presence management application, respectively.

14. The method of claim 13, wherein each of said voice over internet protocol abstraction, instant messaging abstraction and presence management abstraction layers accommodates a plurality of specific features and actions for a plurality of technology-specific solutions.

15. The method of claim 14, wherein each of said voice over internet protocol abstraction, instant messaging abstraction and presence management abstraction layers translate between a general set of features and actions for an application and a specific set of features and actions for a specific solution.

16. The method of claim 15, wherein said translation occurs automatically between networks without additional input from a user.

17. A computer readable medium having a set of instructions for execution by a computer, said set of instructions comprising:
a communication framework interface routine proving a general set of features and actions to facilitate access to a plurality of healthcare enterprise communications applications; and
a plurality of communication abstraction layers accessible via said communication framework interface, each of said plurality of communication abstraction layers defining interface functionality for a communication application.

18. The computer readable medium of claim 17, wherein said communication framework interface routine transmits data from a first system to a second system, said data formatted according to at least one of a first format and a first protocol, said communication framework interface routine selecting an abstraction for said data from among said plurality of communication abstraction layers according to a function of the first system and the second system, said communication framework interface routine abstracting at least one of the first format and first protocol for said data according to the selected abstraction to form abstracted data in at least one of a general format and general protocol for receipt and translation by the second system.

19. The computer readable medium of claim 18, wherein the data comprises at least one of healthcare data, one or more application commands, and an application.

20. The computer readable medium of claim 17, wherein said plurality of communication abstraction layers provide one or more of a voice over internet protocol abstraction, instant messaging abstraction and presence management abstraction layers to translate between a general set of features and actions for an application and a specific set of features and actions for a specific solution, said translation occurring automatically between networks without additional input from a user.
